(19) United States
(12) Patent Application Publication
(10) Pub. No.: US 2026/0215675 A1
(43) Pub. Date: Jul. 30, 2026

(54) Device and System for Initiating an Entoptic Phenomenon That Can be Perceived by a Patient

(71) Applicant: Universität Rostock, Rostock (DE)

(72) Inventors: Rudolf Friedrich Guthoff, Rostock (DE); Niels Grabow, Rostock (DE); Thomas Stahnke, Sande (DE); Wolfram Schmidt, Rostock (DE); Carsten Tautorat, Rostock (DE); Thomas Lips, Kleinmachnow (DE); Kerstin Lebahn, Rostock (DE)

(21) Appl. No.: 19/139,403

(22) PCT Filed: Dec. 13, 2023
(86) PCT No.: PCT/EP2023/085708

§ 371 Date: Jul. 5, 2025

(30) Foreign Application Priority Data
Dec. 14, 2022 (DE) 102022133271.3

(51) Int. Cl. A61B 3/00; A61B 3/12

(52) U.S. Cl. CPC A61B 3/0008; A61B 3/0025; A61B 3/0033; A61B 3/0091; A61B 3/12

(57) ABSTRACT

A device (1) for initiating an entoptic phenomenon perceivable by a patient, a system with such a device (1) as well as a data processing unit (12) providing the patient with specific information relating to the use of the device as well as a functional status of his/her retinal vessels are described.

The device (1) described is characterized in that a housing (4) with a contact surface (6) is provided, which can be placed on the patient's head (8) in the region of the eye (7) in such a way that light emitted by the lighting unit (5) falls at least partially onto the retina (2), wherein the lighting unit (5) is configured to emit light at different times along various beam paths in such a way that the emitted light strikes the retina (2) at various points when the contact surface (6) is in place.

### CROSS REFERENCE TO RELATED APPLICATIONS

This application is the U.S. national stage of International Application No. PCT/EP2023/085708, filed on 2023-12-13. The international application claims the priority of DE 102022133271.3 filed on 2022 Dec. 14; all applications are incorporated by reference herein in their entirety.

### BACKGROUND

The invention relates to a device for initiating an entoptic phenomenon perceivable by a patient. The invention also relates to a system with such a device as well as a data processing unit, which provides the patient with specific information relating to the use of the device as well as a functional status of his/her retinal vessels. The device described comprises a handle and a housing, in which a lighting unit is arranged and which has a contact surface, so that the device can be placed on the patient's head in the region of the eye and aligned by the patient in a targeted manner.

Different methods are known from the state of the art, using which a non-invasive, indirect observation of the retinal vessels, in particular the Purkinje vascular figure and the macular chagrin, is made possible by triggering an entoptic perception. The main application of the aforementioned methods lies in the early detection and monitoring of diabetic retinopathy as well as the assessment of postoperative visual acuity in the case of media opacity.

The entoptic Purkinje tree test can be used to view the retinal blood vessels of the patient's own eye. In normal vision, the shadows cast by the retinal vessels are not perceived due to local adaptation. However, light projected through the sclera into the eye, bypassing the optical system, produces briefly perceived shadows of the retinal vessels on other retinal receptors. In this context, FIG. 5 shows a sketch of the “Purkinje tree”. This creates an image of the retinal vessels in an entoptic manner, which, however, quickly fades if the light source is not moved. The vessels are perceived in the form of an enlarged image that represents a complex and branched vascular tree, the “Purkinje tree”. FIG. 5 also shows the special structure of the vessel-free macula with its so-called macular chagrin. The macular chagrin is perceived by the patient in the vessel-free area of the retina as numerous glittering points of light. These points of light appear when a spot of light is moved on the sclera in parallel to the limbus and are presumably caused by light reflections on the boundary surfaces of the foveal cone mosaic. The macular chagrin enables a qualitative functional test of the macula.

The Purkinje vascular figure is an entoptic phenomenon consisting in the perception of the retinal vascular figure as a shadow on the retina. The vascular figure can be observed when a small light source is moved back and forth on the sclera in parallel to the limbus. The retinal blood vessels are normally not subjectively perceptible, since their shadow image on the retina is fixed and is therefore erased by local adaptation. If the light source is moved, the retinal vessels are imaged on changing retinal locations, so that no local adaptation occurs. The Purkinje vascular figure can also be perceived in case of media opacities in the area of cornea and lens.

The principles of the entoptic perception of retinal vessels are generally well known and described in detail in the scientific literature.

What is important for the technical solution described for self-examination of the functional status of the retinal vessels is that the progression of diabetes in diabetic patients is to be determined by observing the vascular supply to the eye at regular intervals. If the examination is carried out by an ophthalmologist, ophthalmoscopic assessment of the retina is the gold standard available, at least if there is no media opacity, such as cataracts, in the eye.

In addition to visual capacity, the monitoring of diabetic retinopathy is important for the detection of cardiovascular diseases. In type 1 and type 2 diabetes patients in particular, an increased risk can already be assumed, if it has been established that the retinal vessels have only a few microaneurysms. However, diabetes patients only notice the development of their microangiopathy very late, since involvement of the center of the retina, which is responsible for a reduction in visual acuity, only occurs late in the course of the disease.

Based on the above, it would be desirable, if a reliable examination of the retinal vessels could be carried out at shorter intervals, in a comparatively simple as well as inexpensive manner and, particularly with regard to medical care in many developing or newly industrializing countries, without the need for an ophthalmologist to be present during the examination.

Clinically, entoptic perception of the retinal vessels and macular chagrin is used to assess the functional status of the retina when severe opacities of the refractive media, such as cornea, lens or vitreous body, are present. In this context, it is known to place a brightly shining penlight with a punctiform light source directly into the open or onto the closed eye of the patient and to move it up and down with a frequency of about 5 Hz to check the potential visual acuity. During this movement, the illumination is either transscleral with the eyelid open, or transpalpebral through the closed eyelid. The process parameters, such as frequency, holding angle or contact pressure, are currently varied by questioning the patient, until the retinal vessels and possibly even the macular chagrin can be perceived by the patient. Alternatively, the moving spot of light is generated by means of a luminous device, in which the patient's head is fixed. Following the initiation of an entoptic perception by the patient, he/she is questioned by the doctor about his/her observation and describes what he/she sees or has seen, resp. The disadvantage of the known examination methods is that simple self-diagnosis is largely ruled out.

In addition, reproducibility of the examination results is often severely limited and depends on the experience as well as the skills of the persons involved, i.e. both the doctor and the patient. Furthermore, the triggered vascular image can no longer be recognized after a few seconds, so that a quantitative examination is also not feasible.

A device for automatically initiating entoptic perception is known from WO 91/16857 A1. The device described has a holder for the patient's chin or forehead, which is intended to achieve reliable fixation of the head during the examination. As soon as the head is in the fixed position, a spot of light is automatically moved over the open or closed eye, thus triggering the patient's entoptic perception. Once again, the patient describes to the doctor the structure and appearance of the retinal vessels as they appear to him/her.

Nevertheless, the success of self-examination and entoptic perception of the retinal vessels with both manual and automated guidance of the spot of light heavily depends on the interaction between doctor and patient. Here, the patient can only control the quality of the perception to a limited extent or not at all, and a stabilized image, on which the patient concentrates to describe his/her retinal vessels, cannot be produced with the known methods.

Based on the technical solutions known from the state of the art as well as the problems described above, the invention is based on the object of creating a handy and easy-to-use device that enables a patient to trigger a reproducible, entoptic perception of his/her retinal vessels and thus perform a reliable self-diagnosis of the functional status of the retina. Operating the device should be simple and require as few operating steps as possible from the patient. By means of the device to be specified, it should thus be possible for a patient to generate a moving spot of light on his/her own retina in a comparatively simple manner and thus obtain reproducible results. In addition, it should be possible for the patient to reliably operate the device him-/herself, without the need for an ophthalmologist to be present.

In addition, a system should be specified, into which a device solving the aforementioned objects can be easily integrated and with which both the generation of an entoptic perception and support for the patient in carrying out a self-diagnosis to determine the functional status of their own retina is ensured. In that, the system should be designed in such a way that a patient can be assisted in a comparatively simple manner both in the operation of the device and in the evaluation of the perceived vascular structures of the retina. In turn, the system used should be usable by the patient without requiring the presence of a doctor for proper use.

It is respectively essential that, on the basis of an entoptic perception of the vascular structures of the retina, a reliable self-diagnosis of the functional status of the vascular structure of the retina is possible in a simple manner and without any other special equipment, and at the same time changes can be detected quickly and easily, so that the patient can consult an ophthalmologist in such a case without any further delay.

### SUMMARY

The object described above is solved with a device according to claim 1 as well as a system according to claim 11. A suitable data processing system for a system for performing a self-diagnosis of the functional status of the retinal vessels is specified in claim 15. Advantageous embodiments of the invention are the subject of the dependent claims and are explained in more detail in the following description with partial reference to the figures.

### DETAILED DESCRIPTION

The invention relates to a device for initiating an entoptic phenomenon on a patient's retina perceivable by the patient, comprising a handle and a housing, in which a lighting unit is arranged. According to the invention, the housing has a contact surface, which can be placed on the patient's head in the region of the eye, in particular on the closed eyelid, in such a way that light emitted by the lighting unit falls at least partially onto the retina, wherein the lighting unit is configured to emit light at different times along various beam paths in such a way that the emitted light strikes the retina at various points when the contact surface is in place. The invention is thus essentially characterized in that the lighting unit can be used to produce a spot of light in at least two different locations on the retina of the patient. In relation to the device, this means that the lighting unit emits light along at least two different beam paths, which are arranged or run, resp., differently relative to the contact surface and the housing of the device. At the same time, the lighting unit is configured such that the housing, which is connected to the handle that can be grasped by the patient, does not have to be moved so that light is emitted along the at least two differently arranged beam paths, and thus a light spot can be generated in at least two locations on the patient's retina.

It is essential for the invention that it is possible for a patient to place the housing of the device, in particular of a hand-held device, on his/her head in the region of the eye, wherein the contact surface preferably encloses the eye to be examined at least in sections, and to generate a spot of light on the retina, which appears to the patient as a moving spot of light, without the patient him-/herself having to move the housing or the device, resp.

In principle, it is conceivable in this context that the lighting unit is designed to generate points of light at different locations on the retina by means of a plurality of light sources that can be switched on and off in a targeted manner and/or that it has a drive for moving a light source or an optical element, in particular a mirror, in order to generate a movable spot of light and apply it to the retina in at least two different locations. In both cases, again, it is not necessary to move the device itself and its housing in order to successively generate a spot of light in at least two locations on the retina.

The invention therefore provides a device using which it is possible for a patient to trigger an entoptic perception of his/her own vascular structure of the retina in a simple, reliable and reproducible manner. A particular advantage of the device according to the invention is that the patient him-/herself can place the device on the eye to be examined and intuitively vary, for example, the contact pressure and angle of incidence of the light emitted by the light source as required, without the need for additional adjustment steps.

According to a particular embodiment of the invention, the lighting unit has a plurality of light sources, which can be switched on and off or are switched on and off in a targeted manner in order to generate a spot of light at different locations on the patient's retina, which is perceived by the patient as a moving spot of light. The at least two light sources are arranged as required within the housing in the area of the lighting unit.

Alternatively, or in addition, it is conceivable to generate a spot of light and/or spots of light that can be moved or is/are moved relative to the retina in at least two locations on the retina by providing at least one servomotor for controllably moving the at least one light source and/or a deflecting mirror for deflecting a light beam emitted by the light source. Advantageously, the device according to the invention is designed in such a way that the spot of light successively generated, at least temporarily, on the retina of a patient in at least two locations has a diameter of 0.5-3 mm after the housing has been placed on the head of the patient in the region of the eye.

According to a particularly suitable embodiment of the invention, focused light sources or light sources arranged behind optical elements for focusing, such as suitable lenses, are used to generate the at least one light beam.

It has also been shown that it is advantageous for a patient, if spots of light, which lie on a circular path on the retina, or a spot of light, which moves along an at least almost circular path across the retina of the patient, are generated one after the other or simultaneously at least for a short time. In order to generate a spot of light, which is perceived by the patient as a spot of light moving along an at least almost circular path on the retina, the light source provided according to the invention has a plurality of light sources and/or suitable movement means are provided in order to direct a light beam along different beam paths onto the retina of the patient in a targeted manner. In a particular embodiment, at least one servomotor and/or a movable deflecting mirror is provided in order to direct the light beam generated by a light source of the lighting unit in a circular movement across the retina, so that it appears to the observer as if the spot of light is moving in a circle across the retina.

According to an alternative embodiment of the invention, the lighting unit has a plurality of light sources, which are arranged in a circle or at least approximately in a circle within the housing. If these circularly arranged light sources are switched on and off one after the other, the patient perceives a spot of light moving in a circle across the retina. In a particularly advantageous manner, the individual light sources are switched on and off in such a way that a so-called fading effect is achieved, whereby a transition of the light phenomena of the individual adjacent light sources is achieved. In this case, a light source is only switched off completely after an adjacent light source has already been switched on. It is also conceivable that the intensity of the light radiation emitted by the individual light sources is varied in a temporal overlap range between the switching on and off of the individual light sources. In this context, it can be advantageous if the intensity of the light emitted by a light source is slowly increased after it is switched on and/or slowly reduced before it is switched off.

Furthermore, a special embodiment of the invention provides that a plurality of light sources of a lighting unit is arranged in a row. Such a row-shaped arrangement of various light sources can be provided as an alternative or in addition to a circular arrangement of several light sources. Using a plurality of light sources arranged in a row, which are switched on and off one after the other or at least partially overlapping in time, it is thus possible to generate a spot of light, which moves in a line, preferably in opposite directions, across the retina of the patient performing a self-diagnosis.

A further particular embodiment of the invention provides that the lighting unit has a plurality of light sources, in particular LEDs, which are arranged in the form of a matrix, i.e. in rows and columns preferably aligned perpendicular to one another. Particularly preferably, such a matrix is square-shaped, so that the same number of light sources is arranged in the rows of light sources respectively aligned perpendicular to each other.

According to a special further development of the invention, it is further provided that the light sources are at least partially switched on and off one after the other. In addition, it is conceivable that the individual light sources are switched on and off again at least partially overlapping in time, wherein different forms of control are possible. The individual light sources could be switched on and off in a fixed or random sequence by means of a suitable controller, wherein it is again conceivable that at least two of the light sources, in particular adjacent light sources, light up at least temporarily at the same time. With an arrangement of a plurality of light sources in the form of a matrix, a spot of light can be generated on the retina of a patient in an advantageous manner by directing light onto the retina of the patient via different beam paths. Here, the device according to the invention, as with another arrangement of the light sources, is designed in such a way that a patient can carry out a comparatively simple self-diagnosis by simply placing the device on his/her face in the region in front of the eye. Since the patient can in turn compare the images he/she perceives with the reference images provided in an advantageous manner, it is possible for him/her to make an assessment in relation to the condition of the retina even without the presence of a practitioner, in particular a doctor. On the basis of this assessment, the patient can then contact or visit a doctor at an early stage for further diagnosis, so that, especially in areas with a low doctor/patient ratio, it is possible to provide comprehensive support for medical pre- or aftercare measures up to a certain degree of accuracy.

In a particular further development of the invention, an operating element is provided, for example in the form of a pressable, slidable or rotary switching element, by the operation of which a controller can be activated to change a sequence in which the light sources are switched on and off, a wavelength and/or an intensity of the emitted light and/or a frequency at which the light sources are switched on and off. According to this embodiment, the device according to the invention thus has both an operating element and a control unit that enables the function of the individual light sources of the lighting unit to be influenced in a targeted manner. This way, it is possible to influence the switch-on and switch-off time of the individual light sources, the wavelength and/or the intensity of the emitted light and/or the frequency with which light is emitted from different light sources, and thus the properties and the type of movement of a spot of light guided across the retina of the patient in a targeted and needs-based manner. If the operating element, the control unit and at least part of the light sources are configured to change the intensity and/or the wavelength, i.e. in particular the color of the emitted light, it is possible for a patient to adjust the brightness and the color of the spot of light moving across the retina in such a way that the spot of light is not perceived as unpleasant, and comparatively strong entoptic perceptions are triggered in a particularly advantageous manner. It has been shown that light with an increased green component, i.e. with wavelengths in a range of around 500 nm, is perceived as particularly pleasant by a patient during self-diagnosis and at the same time triggers the required entoptic perceptions.

The invention also relates to a system for carrying out a self-diagnosis to determine the functional status of the vascular structure of a retina. It is likewise possible to use the system to detect retinal diseases and/or monitor the course of a disease as part of a self-diagnosis.

The system according to the invention comprises a device according to at least one of the embodiments described above, a data processing unit configured to provide retina-, device- and/or disease-specific information, and an output unit by means of which the information provided can be output to an operator, in particular the patient, carrying out a self-diagnosis at time intervals.

It is particularly advantageous if the data processing unit has at least one data storage unit and/or has a data interface configured to exchange data containing the required or desired information uni- or bidirectionally with an external data storage unit. In that, it is initially conceivable that the patient is provided with the desired information quickly and reliably before, during or after the procedure, in particular without having to visit a doctor for that.

According to a special further development of the invention, data is alternatively or additionally stored in the internal or external data storage, which contains information about properties of a retina, device- or equipment-specific information, such as operating instructions and/or information about vascular structures of certain retinal diseases. It is likewise conceivable that data is stored on the internal or external data storage by a user, in particular a patient who is performing or has performed a self-diagnosis. This is an advantageous way of documenting observations made by the patient or even creating a kind of diary of the results of the self-diagnoses performed.

It is particularly advantageous, if the data processing unit described above is part of a tablet computer, a mobile phone and/or a smartphone in at least one of its embodiments. Alternatively, it is conceivable that the data processing unit is at least partially integrated into a device for self-diagnosis of the functional status of the retina according to an embodiment as described above and can be connected to a tablet computer, a mobile phone and/or a smartphone via at least one suitable interface for further data communication. In this context, it is conceivable in a particularly advantageous manner that a device designed according to the invention for initiating an entoptic perception of the vascular structures of the retina is used in a system together with a tablet computer or a smartphone, on which a special application (app) is implemented.

An app designed in a suitable manner and implemented on a tablet computer or a smartphone is preferably configured such that at least one piece of information about properties, the operating status and/or the operating options of the device according to the invention can be output to a patient before, after or during self-diagnosis via an output unit of the tablet computer or the smartphone, in particular via a speaker or a display, in response to a corresponding request. In this context, it is conceivable that information on the appropriate use or operation, resp., maintenance and/or servicing of the device according to the invention can be output in a targeted and needs-based manner in response to a request. It is likewise conceivable that, by using the application implemented in the data processing unit, vascular structures of the retina can be shown on a display, providing a patient with more detailed information about both healthy vascular structures and possible clinical pictures. In a particularly preferred manner, the application is designed and implemented on a tablet computer or a smartphone such that it provides a patient performing or wishing to perform a self-diagnosis with a device according to the invention, which is preferably designed as a handheld device, with information about operating steps, possibly perceptible vascular structures of the retina and/or about specific clinical pictures.

According to a particularly suitable embodiment, certain vascular structures, the data of which is stored in an internal or external data storage, are graphically shown to the user on a display, wherein the patient also preferably has the option of hiding or showing structural elements and/or certain parts of the vascular structures initially shown. It is likewise also conceivable, for example when perceiving an aneurysm in the area of the retina, that the patient places a marker at a corresponding point in the vascular structure shown on a display, which represents the aneurysm perceived by him/her on the retina. In a special further development of the invention, the vascular structures changed and/or generated by the patient on a display or the data on which they are based can in turn be stored in an internal or external data storage, wherein the stored data sets are preferably provided with a time stamp.

This way, it is possible not only to carry out a comparatively simple self-diagnosis, but also to document the course of a disease. The invention thus also relates to a data processing unit for a system according to one of the embodiments described above with an implemented computer program product, in particular a special application (app), which is configured to request specific data containing retina-, device- and/or disease-specific information from a plurality of data sets as a function of a request signal as well as to effect the transmission of the data to an output unit and the output of the requested information via the output unit, in particular a display. Furthermore, the implemented computer program product is designed and implemented on the data processing unit such that the patient can retrievably store data changed and/or generated by him/her on an internal data storage of the data processing unit or an external data storage.

A further special embodiment of the invention provides that a system according to at least one of the previously described embodiments, in particular the data processing unit or a tablet computer, a mobile phone or a smartphone with a data processing unit further developed according to the invention, has at least one interface enabling uni- or bidirectional data communication with an external data processing unit. The system according to the invention further developed in this way is advantageously configured such that it can exchange data with a telemedicine system and/or can be integrated into a telemedicine system. This way, information about the patient, about the patient's state of health, about self-diagnoses carried out and/or results can be made available directly to a telemedicine system, wherein the information is preferably accessible by an authorized physician. In particular, this way, vascular structures entoptically perceived by the patient can be transmitted to an ophthalmologist, whereupon he/she is in turn able to send suitable information to the data processing unit used by the patient and thus to the patient. It is therefore comparatively easy and quick to transmit information about the functional status of the vascular structures of a retina, in particular any changes that have taken place, to an ophthalmologist providing treatment, and to receive instructions from the ophthalmologist and/or initiate further steps just as quickly. All this is possible without the patient having to physically visit the ophthalmologist. In this respect, the invention not only ensures regular and early diagnosis, but also enables reliable monitoring of the functional status of the retina even without the presence of the ophthalmologist and is therefore suitable for use in areas with a low patient/ophthalmologist ratio, as is sometimes the case in developing and newly industrializing countries.

### BRIEF DESCRIPTION OF THE DRAWINGS

In the following, the invention is explained in more detail, without limiting the general idea of the invention, with reference to the figures, using specific embodiments. In that, identical elements are marked with the same reference signs. Therein:

FIG. 1: shows a schematic diagram of the light-spot stimulation known from the state of the art for initiating an entoptic perception in the left and the right human eye;

FIG. 2: shows a device designed according to the invention for initiating an entoptic perception of the vascular structures of a retina;

FIG. 3: shows a system consisting of a device according to the invention and a suitably designed data processing unit;

FIG. 4: shows a schematic representation of the entoptic self-diagnosis by means of a system according to the invention; as well as

FIG. 5: shows a sketch of a Purkinje vascular figure (Purkinje tree structure) with macular chagrin.

### DETAILED DESCRIPTION OF THE PREFERRED EMBODIMENTS

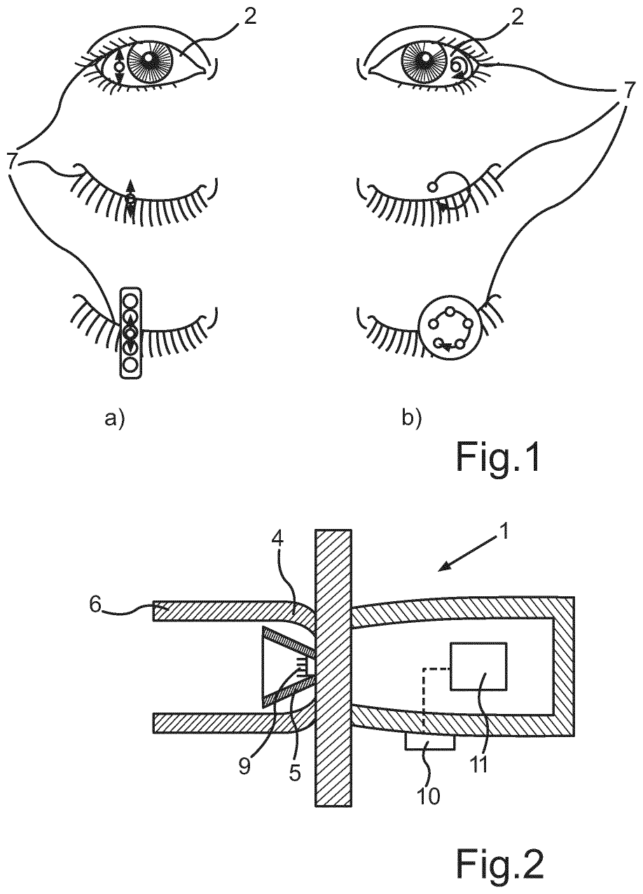

FIG. 1 schematically shows a light-spot stimulation for initiating an entoptic perception on the left and the right human eye 7 of a patient, as is known from the state of the art. For stimulation, a penlight is used to generate a spot of light that moves either linearly back and forth or in a circle over the open or closed eye 7, so that the corresponding light phenomenon on the retina 2 is perceived by the patient. According to the embodiment in FIG. 1a), the penlight is moved up and down at a preferred frequency of approx. 5 Hz.

FIG. 1b) shows an alternative embodiment, in which the practitioner moves the penlight in a circular motion over the eye 7, so that the spot of light moves in a circular motion across the patient's retina 2.

With the procedure described above, the practitioner can trigger the patient's entoptic perception of the Purkinje vascular figure and the macular chagrin. This method is primarily used for early detection and monitoring of diabetic retinopathy as well as for the assessment of postoperative visual acuity in the case of media opacity. However, the problem with this method is that the entoptic perception of the retinal vessels 20 is highly dependent on the interaction between doctor and patient and that the patient can only control the quality of the perception to a limited extent or not at all. In particular, it is not possible to generate a stabilized image, on which the patient can concentrate to describe his/her retinal vessels 20. As will be explained below, the invention solves this problem and, in particular, enables the patient to make a reliable and quick self-diagnosis without the need for the presence of an ophthalmologist.

FIG. 2 shows a device 1 according to the invention for a non-invasive, direct observation of the retinal vessels 20, in particular the Purkinje vascular figure as well as the macular chagrin, by means of entoptic perception. The device 1 illustrated comprises a housing 4, a handle 3, as well as a lighting unit 5, which is arranged inside the housing 4. A contact surface 6 is arranged in the front area of the housing 4, which surrounds the lighting unit 5 on the periphery and which can be positioned on the head 8 of a patient such that the contact surface 6 surrounds the eye 7 to be examined. In that, the contact pressure as well as the alignment of the device 1 can be adjusted by the patient as required.

According to the embodiment shown in FIG. 2, the lighting unit 5 has a plurality of light sources 9, which are arranged in a circle and/or in a row within the housing 4. Upon using this device 1, the individual light sources 9 are switched on one after the other and later switched off again, so that light originating from the various light sources 9 strikes different points of the retina 2 to be examined spaced apart in time.

In an alternative embodiment of the invention, a servomotor is provided, which enables a targeted movement of a light source 9 or a deflecting mirror, so that the emitted light is also emitted in this manner via different beam paths and ultimately strikes various points of the patient's retina 2, so that he/she perceives a spot of light moving across the retina 2.

Different modes of operation can be set by the patient with regard to the type of switch-on and switch-off processes, the movement of a light source 9 or a deflecting mirror, the intensity of the emitted light as well as the light color. If a movable light source 9 or a movable deflecting mirror is used, then two modes of operation can be set in relation to its movement, so that the spot of light is either moved back and forth or at least approximately circularly across the patient's retina 2.

With the device 1 shown in FIG. 2 in the form of a hand-held device, which comprises a lighting unit 5 with a plurality of light sources 9, the patient can trigger the entoptic perception of the Purkinje vascular figure in him-/herself. Here, using the device 1 in the mode of operation, in which a spot of light is moved in a circle across the retina 2, is advantageous, since tests have shown that this enables optimum perception of the retinal vessels 20.

According to the invention, a device 1, preferably in the form of a small portable hand-held device, for initiating entoptic perceptions is provided through the use of comparatively simple technical means with simultaneous miniaturization. The advantage here is that the patient him-/herself can place the device with its contact surface 6 on the eye 7 to be examined and usually performs self-adjustment completely independently. For adjustment, the patient intuitively varies the contact pressure in particular and, especially with devices 1 that have several light sources 9 that cannot be moved by a servomotor, the angle of incidence, until the vascular structures appear optimal. If he/she cannot achieve this, the device 1 additionally allows individual adjustment of the light stimulation by varying the light intensity, the light color, the shape of the light beam and/or, depending on the design of the lighting unit, the movement of the light beam.

With the device 1 shown in FIG. 2, a light beam is generated and emitted, so that the spot of light striking the retina 2 at different points has a diameter of approximately 0.5 to 3 mm. The focus of the light beam, and thus the diameter of the spot of light in the plane of the retina 2, can be changed by using optical lenses or movable lenses arranged downstream of a light source 9 as required.

Using the invention, a moving spot of light is thus automatically generated on the patient's retina 2, as was previously only possible, if a treating physician manually moved a penlight over an eye 7 in a suitable manner. In contrast to the methods known from the state of the art, the device 1 shown in FIG. 2 allows the light beam to be directed through the closed eyelid 7 (transpalpebral) onto the retina 2 in a standardized, reproducible manner with a defined wavelength and standardized circular stimulus application, as also shown in FIG. 1b). This bypasses the local adaptation of the retina and generates a stable optical perception.

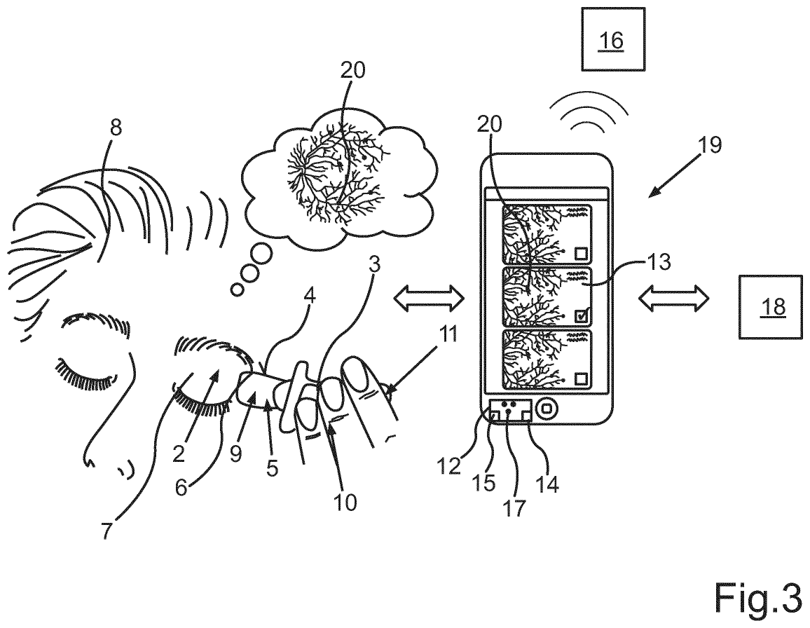

FIG. 3 shows a schematic representation of a system for suitably carrying out a self-diagnosis of the functional status of the retinal vessels 20, which, on the one hand, comprises a device 1 designed according to the invention and, in addition, a specially equipped data processing unit 12. In that, the data processing unit 12 is able to store information about the device 1, such as different operating settings or an operating manual, properties and representations of structures of the retinal vessels 20 and/or information about relevant clinical pictures, and to output this information on request by the user via an output unit 13 in the form of a display. It is likewise possible for the patient to store the results of his/her self-diagnosis in an internal data storage unit 14 of the data processing unit 12. An interactive procedure is preferably possible here, in which the patient compares the vascular structures displayed to him/her with the structures he/she perceives and can actively show or hide substructures. It is also possible for the patient to set a marker for aneurysms perceived during the self-diagnosis. In addition, the patient can keep a kind of diagnostic diary and thus document the results he/she perceives, so that a progress review is also possible.

The data processing unit 12 can also be connected to a telemedicine system 18 via a suitable interface 15, so that it is possible, for example, for an ophthalmologist to retrieve the information stored by the patient and, based on the findings derived therefrom, to provide the patient with recommendations for action or therapy, in particular for retrieval via the data processing unit 12. In order to be able to implement the functions described above, a computer program product 17 or an application (app) is implemented on the data processing unit 12, which, for example, is part of a smartphone 19 or a tablet computer, which enables the patient to operate the system easily and safely.

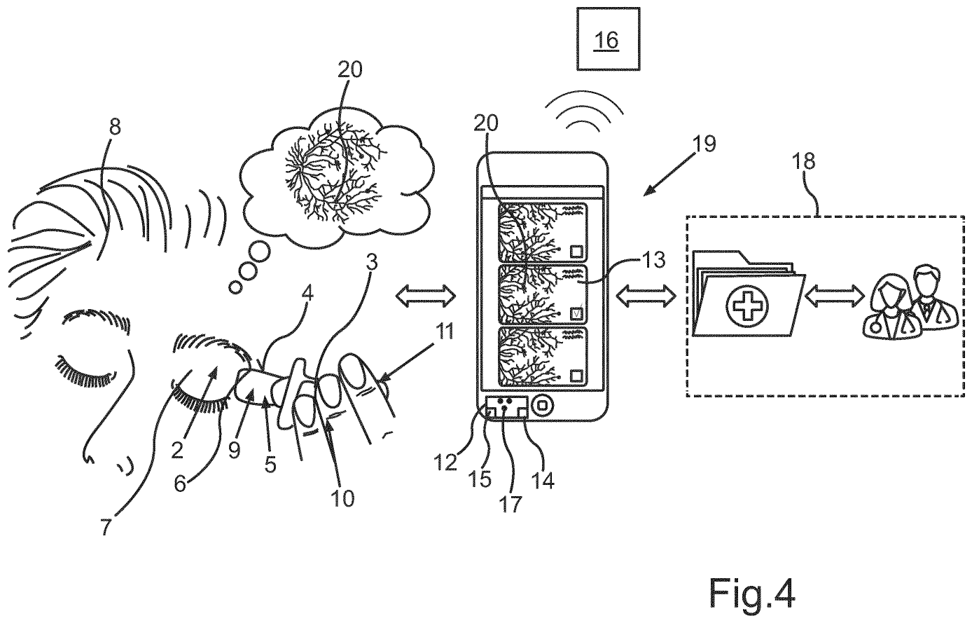

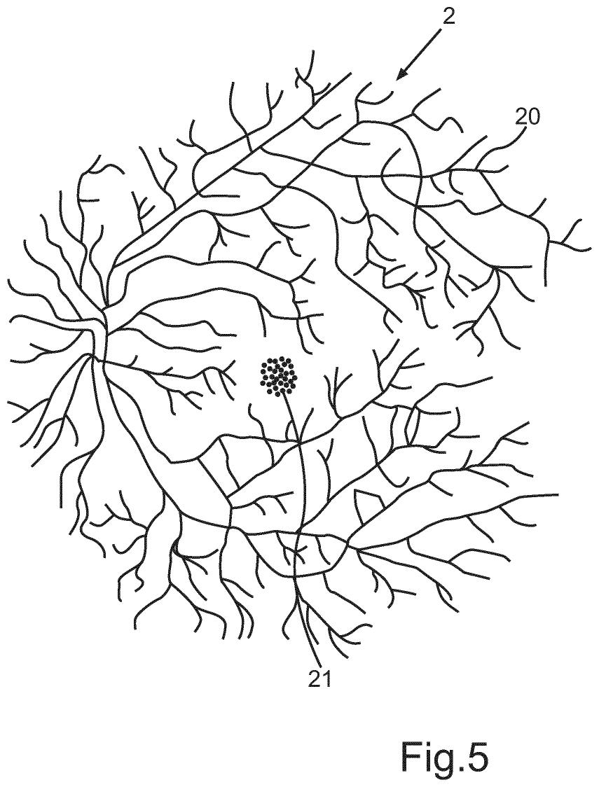

In this context, FIG. 4 additionally shows a schematic representation of performing an entoptic self-diagnosis by means of a system according to the invention. The patient uses the device 1 according to the invention, here in the form of a hand-held device, for self-diagnosis of the functional status of his/her retinal vessels 20. The patient him-/herself holds the device 1 against the closed eye 7 to be examined. By optimizing the contact pressure and holding angle, the patient corrects the position of the device 1 until a stabilized entoptic perception of the vascular structures is achieved. As required, various stimulation parameters, such as the intensity of the light or the light color, can be varied via an operating element 10 on the handle 3, the actuation of which causes a control signal to be generated by a control unit 11 or controller.

For description and documentation of the subjective perception of the retinal vessels 20, the user is supported by the use of an assisting computer program product 17, in this case an application (app) implemented on a smartphone 19. According to the embodiment described, the patient can, for example, display instructions for operating the device 1, typical example images for vascular structures or training and explanatory videos, and also document his/her own results. Furthermore, the computer program product 17 is configured such that the patient can undergo training in regards to the self-diagnosis to be carried out by him/her, which provides sufficient expert knowledge and is preferably undertaken using animated example images in the form of a map-like representation of retinal vessels 20. Pathologies detected by the patient, such as microaneurysms, can be added or removed with a click in a standardized representation of a typical vascular structure of the retina. The computer program product 17 implemented on the smartphone 19 thus offers the possibility of documenting the course of the disease.

Furthermore, an interface 15 for data exchange with a telemedicine system 18 is provided. Via this interface, the computer program product 17 can be used to exchange the patient's health data with a telemedicine system 19 and even enable external access by medical staff or a treating physician to the data stored in a data storage 14 of the smartphone 19 or an external data storage 16 in order to clarify the patient's state of health. A major advantage of this is that the patient's willingness (adherence) to actively participate in therapeutic measures increases and the number of visits to the doctor can be reduced, if necessary. In this context, it is conceivable that a treating ophthalmologist could routinely check the state of health of his/her patients by retrieving the patient data. During a visit to the doctor, the data generated by the patient him-/herself can then be evaluated as a whole together with the current findings.

The system according to the invention thus makes it possible in a special way to document, evaluate and use results generated by the patient in the course of entoptic self-diagnosis. Using the computer program product 17 implemented on a smartphone 19, a matrix is generated on the display 13 of the smartphone 19 after self-diagnosis has been performed by the patient, on which the pathological changes perceived by the patient are documented and objectified. The information entered by the patient supports the staging of microangiopathy in diabetic retinopathy, for example, and may help to detect progression. This offer of cooperation by the patient is expected to significantly increase adherence and inform the treating ophthalmologist about progression at an early stage.

A table-top device with interchangeable devices 1 was developed for experiments and initial clinical studies. In order to stimulate entoptic perception, the devices each have lighting units 5 with several light sources 9. To test a suitable arrangement of the light sources 9, the device 1 can be exchanged. Four devices 1 with various at least almost circular arrangements of several light sources 9 and one device 1 with an arrangement in a row were examined. The arrangement of several light sources 9 in a row within the housing 4 of the device 1 simulates the mode of action of a moving light beam, e.g. a penlight, which is moved back and forth over the patient's eye 7, as shown in FIG. 1a).

The table-top device contains electronics and a programmable microcomputer that enables the wired electrical control of the device 1 respectively used. Operating elements on the table-top device allow the commissioning and variation of illumination parameters, such as light intensity, stimulation pattern and stimulation speed, in order to generate a stable shadow image of the retinal vessels 20 in the observer.

In general, light sources 9 preferably are light-emitting diodes with a small beam angle, which is particularly preferably less than 20°. The light intensity can preferably be adapted to the needs of a patient performing self-diagnosis by pulse width modulation. In order to be able to adjust the light color, i.e. the wavelength of the light respectively emitted, devices 1 were used that employ lighting units 5 with light sources 9 emitting light at different wavelengths. Furthermore, light-emitting diodes with white light were tested, although their characteristic wavelengths dominate in the blue and yellow range of wavelengths. A pleasant entoptic perception was observed by the test persons predominantly with green light-emitting diodes, which emit light with a wavelength of around 525 nm, which is why this color of the emitted light appears to be particularly advantageous. The preferred light-emitting diodes have a standard LED housing made of plastic with integrated optical lenses for a focused beam angle of around 15°, wherein the LED housing has a diameter of 3 mm.

For the devices 1 with light sources 9 arranged in a circle, a rotating running light with a fading transition (fading effect) of the adjacent light sources 9 was determined as the preferred stimulation pattern in the preliminary test in order to simulate a continuous movement of a punctual light source 9. Here, the individual light sources 9 were thus switched on and off in such a way that light was emitted from at least two adjacently arranged light sources 9 for at least a certain period of time. The individual controller 11 of the individual light sources provided by the design allows this type of commissioning. A preferred direction of rotation, i.e. either counterclockwise or clockwise, was not determined.

For the device 1 having LEDs arranged in a row as the light sources 9, it is preferable to realize a back and forth movement of the spot of light. For the evaluation of the test results, the radius of curvature of the eye was assumed to be 15 mm.

### LIST OF REFERENCE NUMERALS

1 Device
2 Retina
3 Handle
4 Housing
5 Lighting unit
6 Contact surface
7 Eye
8 Head
9 Light source
10 Operating element
11 Controller
12 Data processing unit
13 Output unit
14 Data storage unit
15 Data interface
16 External data storage
17 Computer program product
18 Telemedicine system
19 Smartphone
20 Retinal vessels

1. A device (1) for initiating an entoptic phenomenon in a patient perceivable by the patient, having a handle (3) and a housing (4), in which a lighting unit (5) is arranged,
characterized in that the housing (4) has a contact surface (6), which can be placed on the head (8) of the patient in the region of the eye (7) in such a way that light emitted by the lighting unit (5) falls at least partially onto the retina (2), wherein the lighting unit (5) is configured to emit light at different times along various beam paths in such a way that the emitted light strikes the retina (2) at various points when the contact surface (6) is in place.

2. The device according to claim 1,
characterized in that the lighting unit (5) has a light source (9) movable by a drive and/or a mirror movable by a drive.

3. The device according to claim 1,
characterized in that the lighting unit (5) has a plurality of light sources (9), which can be switched on and off independently of each other.

4. The device according to claim 3,
characterized in that the light sources (9) are arranged circularly or at least approximately circularly within the housing (4).

5. The device according to claim 3,
characterized in that the light sources (9) are arranged in a row within the housing (4).

6. The device according to claim 3,
characterized in that the light sources (9) are arranged in the form of a matrix, in particular in rows and columns perpendicular to each other.

7. The device according to claim 6,
characterized in that the light sources (9) are arranged in the form of a rectangular, in particular a square matrix.

8. The device according to claim 6,
characterized in that the light sources (9) can be switched on and off at least partially by means of a controller in such a way that at least two light sources (9), preferably arranged adjacent to each other, light up together at times.

9. The device according to claim 3,
characterized in that an operating element (10) is provided, by the operation of which a controller (11) for changing a sequence, in which the light sources (9) are switched on and off, and/or a frequency, with which the light sources (9) are switched on and off, can be activated.

10. The device according to claim 1,
characterized in that an operating element (10) is provided, by the operation of which a controller (11) for changing an intensity and/or a wavelength of the emitted light can be activated.

11. A system for detecting a retinal disease comprising a device (1) according to claim 1, a data processing unit (12) for providing retina-, device- and/or disease-specific information and an output unit (13), via which the information provided can be output.

12. The system according to claim 11,
characterized in that the data processing unit (12) comprises a data storage unit (14) and/or has a data interface (15), which is configured to uni- or bidirectionally exchange date with an external data storage (16).

13. The system according to claim 11,
characterized in that the data processing unit (12) is part of a mobile phone, a smartphone or a tablet computer.

14. The system according to claim 11,
characterized in that in the data processing unit (12) a computer program product (17) is implemented, which is configured such that, in response to an input of the patient, information can be provided, data can be stored in an internal data storage of the data processing unit, data can be transferred to an external data storage and/or data can be uni- or bidirectionally exchanged with a telemedicine system.

15. A data processing unit (12) for a system according to claim 11 with an implemented computer program product (17), which, depending on a request signal, is configured to select a specific data set from a plurality of data sets containing retina-, device- and/or disease-specific information and to effect a transfer to an output unit (13).

16. A computer program product configured to cause an interaction between the patient and the system according to claim 11 and/or between the patient and a data processing unit according to claim 15.
